(19) United States
(12) Patent Application Publication
(10) Pub. No.: US 2005/0027460 A1
(43) Pub. Date: Feb. 3, 2005

(54) Method, program product and apparatus for discovering functionally similar gene expression profiles

(76) Inventors: Bhooshan Prafulla Kelkar, Flower Mound, TX (US); Tanveer F. Syeda-Mahmood, Cupertino, CA (US); Gregor M. Meyer, Loeningen (DE)

Correspondence Address: International Business Machines Corporation;Intellectual Property Law Dept, 8501 IBM Drive, Charlotte, NC, 28262-4333, US

(21) Appl. No.: 10/629,448

(22) Filed: Jul. 29, 2003

(51) Int. Cl. G06F 19/00; G01N 33/48; G01N 33/50

(52) U.S. Cl. 702/20

(57) ABSTRACT

Genes to be compared are listed by their gene expression profiles and processed with a similar sequences algorithm that is a time and intensity invariant correlation function to obtain a data set of gene expression pairs and a match fraction for each pair. A threshold match fraction is chosen and a null set is created to hold indices of genes accounted for. Genes are then assigned to clusters by match fraction value if they have a match fraction greater than the threshold. Genes are then removed from clusters if they are represented in more than one cluster by removing a first gene from a cluster when another cluster has another gene with a higher match fraction with the first gene. When the difference between maximum match fraction values for pairs including a first gene in a first cluster and the first gene a second cluster is small, the first gene may be removed from the first cluster even when another gene in the first cluster has a higher match fraction with the first gene than the first gene has with a third gene in a second cluster. This occurs when the number of similar subsequences for the pair including the first gene in the first cluster is higher than the number of similar subsequences for the pair including the first gene in the second cluster.

### FIELD OF THE INVENTION

The present invention relates generally to the field of data analysis methods, systems and apparatus, sometimes referred to as data mining.

### DESCRIPTION OF THE PRIOR ART

In today's systems, there is a severe shortage of advanced data analysis software to search for information in large genome data sets. Current statistical and data mining tools cannot adequately address the needs of scientists that want to find answers to complex questions in genome data sets. Now that the human genome has been sequenced, a greater challenge faces the scientists: to use the information being populated in aenome databases worldwide for improved disease diagnosis and drug discovery. With advances in sequencing techniques, increasingly large amounts of data is becoming available on a worldwide basis as a combination of public and private genome databases. It has been estimated that a single genome may require as much as 300 Terabytes of trace files. With the genomes of several organisms completely sequenced, interest within bio-informatics has shifted from sequencing to learning more about the genes encoded in the sequence and their functions. Specifically, scientists would like answers to questions such as
1. Are gene expression levels in these samples indicative of cell proliferation?
2. How does the complex interaction over time between genes control cellular differentiation during development, aging and disease?
3. Are there genes of similar function?
   Specifically, discovering functionally similar genes is an important aspect of drug discovery as well as disease diagnosis. Current methods of discovering functional similarity in genes use only the intensity of expression. However, the intensity of gene expression can vary with time and follows a specific pattern. For example, progression through the eukaryotic cell cycle is known to be both regulated and accompanied by characteristic periodic fluctuations in the expression levels of numerous genes.
   This problem or issue of finding similarities in gene expression data is typically done using time dependent clustering by many vendors in this market. As an example, ArrayScout from Lion BioSciences or clustering from SpotFire. This analysis is only intensity-based.

WO0237102A2 “Methods for Analyzing Dynamic Changes in Cellular Informatics and Uses Therefor” by Huang and Ingber describes analysis of dynamic changes in cellular processes and representing cellular processes as dynamic signatures or phase portraits. The signature is based upon time dependent molecular changes that are associated with a transition between distinct stable cellular behavioral states.

WO0134789A2 related to gene expression clustering by statistically significant connections.

U.S. Pat. No. 6,420,108 relates to a computer aided display for comparative gene expression.

US 2002/0019704 is a method for analyzing a plurality of sets of values associated with a plurality of genes to identify those genes whose associated values differ by an amount of statistical significance.

U.S. Pat. No. 6,185,561 relates to organizing expression information in a way that facilitates data mining.

### SUMMARY OF THE INVENTION

The present invention provides a method and programmed means for clustering genes having potential functional similarity by a comparison of their time varying gene expression profiles.

The temporal expression patterns of large number of genes are known to exhibit some degree of order across a tissue. Therefore, a match of the gene expression profiles using both time and intensity information is better at detecting functional similarity than using intensity alone.

According to the instant invention, two temporal sequences are similar and can be placed in the same cluster if they have enough non-overlapping time-ordered pairs of sub-sequences that are similar.

It is an advantage of our invention that functional similarity between portions of gene expression profiles can be clustered, thereby characterizing similarity between genes in one or more phases of the cell cycle.

Another advantage of our invention is that it can cluster all similar genes without a linear search of the genome database through a fast multidimensional index structure.

These and other advantages of the invention which will become clear upon reading the following description of a preferred embodiment are obtained by novel processes of clustering the result of several methods of signal matching in signal processing, such as correlation.

### BRIEF DESCRIPTION OF THE DRAWINGS

FIG. 1 is a block diagram including an alternate embodiment of the invention.

FIG. 2 is a block diagram of the invention.

FIG. 3 is a graphic presentation of two gene expression profiles g1 and g2.

FIG. 4 is a graphic presentation of two gene expression profiles g3 and g4.

FIG. 5 is a graphic presentation of two gene expression profiles g5 and g6.

FIG. 6 is a graphic presentation of two gene expression profiles g7 and g2.

FIG. 7 is a graphic presentation of two gene expression profiles g8 and g9.

FIG. 8 is a block diagram illustrating a computer architecture of the preferred embodiment.

### DESCRIPTION OF THE PREFERRED EMBODIMENT

A method and programmed means is disclosed for discovering functional similarity between portions of gene expression profiles, to cluster all similar genes without a linear search of the genome, thereby characterizing similarity between genes in one or more phases of a cell cycle. Our preferred embodiment uses a time and intensity-invariant correlation function such as that described by R. Agriawal, K. Lin, H. S. Sawhney, K. Shim: “Fast Similarity Search in the Presence of Noise, Scaling, and Translation in Time-Series Databases”, Proc. Of the 21st Int'l Conference on Very Large Databases, Zurich, Switzerland, September 1995. Specifically, we employ the similar sequences algorithm embodiment of the above described correlation function in Intelligent. Miner for Data (™ IBM Corp.), which was designed for business intelligence, against time varying gene expression data.

The method of the invention uses the time and intensity invariant correlation function of the IBM tool to find matches of gene expression profiles using both time and intensity information, which is better at detecting functional similarity than using intensity information alone. The output of Intelligent Miner is a data set of gene expression pairs with the match factor and number of subsets used to compare each pair. A threshold match factor is chosen and genes are listed in clusters by their match fractions. Genes are then removed from all except the cluster with the highest match fraction. Any genes not already in a cluster are added to a cluster which includes a gene that has a highest match fraction with the added gene.

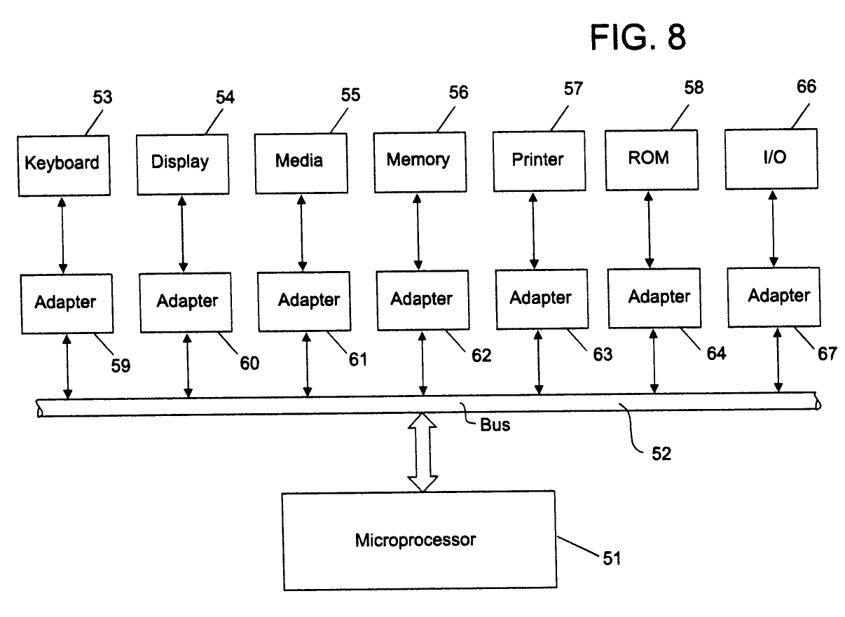

Referring now to the drawings, and first to FIG. 8 for the purpose of describing the present invention in the context of a particular embodiment, a typical computer architecture is shown. The present invention may also be used in any digital computer architectures, including personal, minicomputer and mainframe computer environments, and in local area and wide area computer networks.

The focal point of the preferred personal computer architecture comprises a processor 51. The processor 51 is connected to a bus 52 which comprises a set of data lines, a set of address lines and a set of control lines. A plurality of I/O devices, memory and storage devices 53-58 and 66 are connected to the bus 52 through separate adapters 59-64 and 67, respectively. For example, the display 54 may be either a CRT or a flat panel display.

The random access memory (RAM) 56 and the read-only memory (ROM) 58 and their corresponding adapters 62 and 64 are included as standard equipment in most computers, although additional random access memory to supplement memory 56 may be added via a plug-in memory expansion option.

Within the ROM 58 are stored a plurality of instructions, known as the basic input/output operating system, or BIOS, for execution by the processor 51. The BIDS controls the fundamental operations of the computer. An operating system such as a windows oriented operating system software available from IBM Corporation, MICROSOFT Corporation or other supplier is loaded into the memory 56 and runs in conjunction with the BIOS stored in ROM 58.

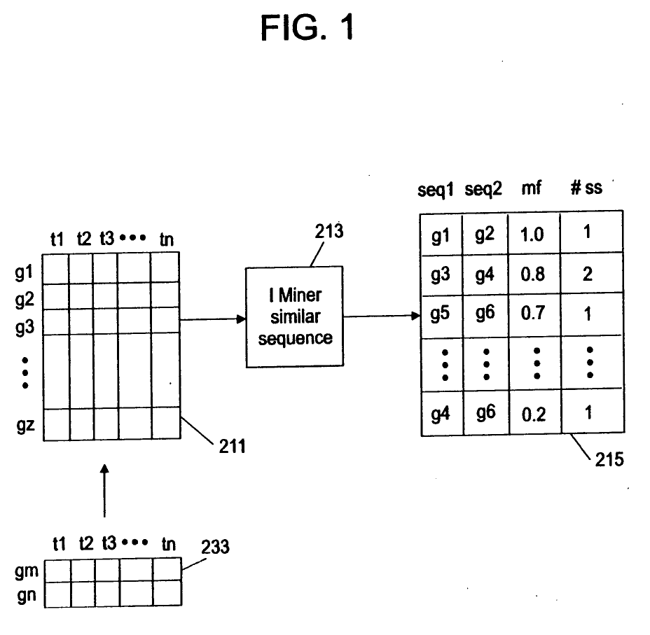

The programs embodying the instant invention as well as other programs such as scientific instrument control programs may also be loaded into the memory 56 to provide instructions to the microprocessor 51 to enable a comprehensive set of tasks, including the gathering of gene expression profiles to be performed by the computer system shown in FIG. 1. An application program including the programs: Intelligent Miner™ IBM Corp., with associated files used in embodying the instant invention, is loaded into the memory 56 and runs in conjunction with the operating system previously loaded into the memory 56 to the correlate the gene expression profiles into groups of functionally similar genes. These programs are contained in media 55 such as a diskette or compact disc or they are part of a communication signal received at a modem or other communications connection version of media 55. Media 55 is connected to bus 52 by an adapter 61 which may be in the form of a communications adapter.

In a computer such as the computer for the system shown in FIG. 8, other input/output devices 66 and an I/O adapter 67 is also provided. These devices are available in many versions and forms including tablets, plotters, touch screens, light pens, joysticks, trackballs, scientific instruments and similar devices.

Computer architecture and components are further explained in The Winn Rosch Hardware Bible, W. L. Rosch, Simon & Schuster, ISBN 0-13-160979-3 (“Rosch”), which is specifically incorporated herein by reference.

Referring now to FIG. 1, the preparatory steps of the method of the invention will be described. The gene expression profiles to be analyzed for similar genes are contained in a data set 211. This data set is provided to a similar sequences algorithm 213 that is a time and intensity invariant correlation function to obtain a data set of gene expression pairs and a match fraction for each pair. The similar sequence algorithm 213 in our preferred embodiment is part of the IBM Program Product, Intelligent Miner for Data (™ IBM Corp.). The data set of gene expression pairs 215 is the output of Intelligent Miner (™ IBM Corp.) and is already organized in descending order of match fraction value.

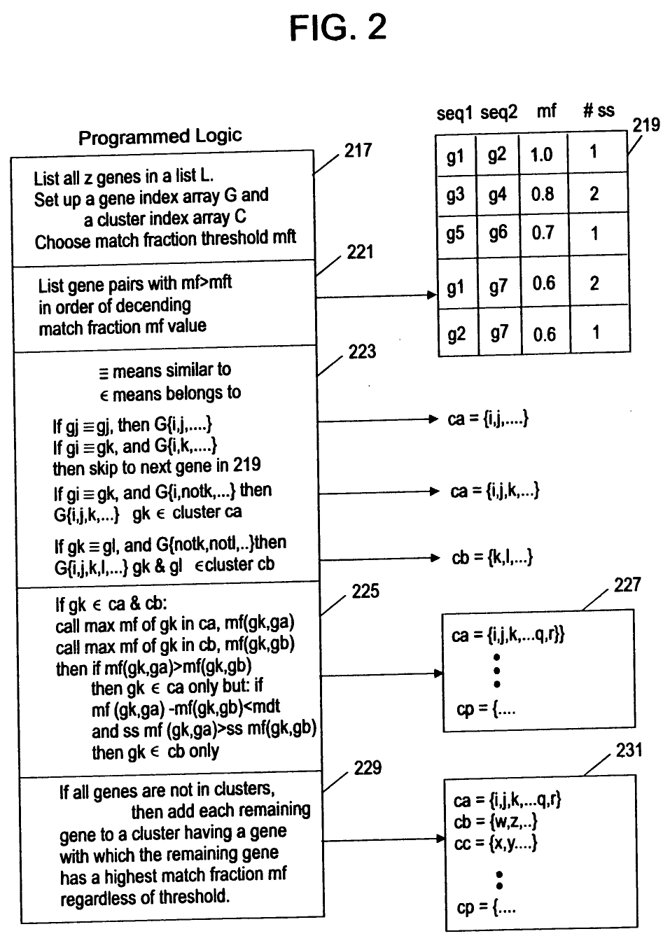

Referring now to FIG. 2, at block 217, list L of all the genes analyzed is generated for control purposes. Likewise a null gene index array G and a null cluster index array C are set up in block 217.

The program product logic means of the invention has a clustering section 223 which lists gene expression pairs in clusters by their match fractions. If gene gi is similar to gene gj, then these two genes are placed in a cluster ca and i and j are added to the gene index array G and to the cluster index array C. The next gene expression pair gi and gk are then examined. If gene gi is similar to gene gk, but i and k are already in the gene index array G then the next gene expression pair is examined. But if gene gi is in the index G but gene k is not, then gene k is placed in cluster ca with i and j by adding k to G and to C as indicated at 223.

The program product of the invention also has means at block 225 for removing a first gene from a cluster cb when the first gene is also in another cluster ca which has another gene with a higher match fraction with the first gene than any of the genes in the cluster cb have with the first gene. When a gene has such a higher match fraction mf with another gene in another cluster ca but the difference between the match fractions is less than a predetermined match difference threshold mdt value such as 5 percent, and the similarity with the other gene comprises more subsequences than the similarity in the cluster cb, then the gene is placed only in the cluster cb and is removed from the another cluster ca. This programmed logic removing means is cycled until all genes are listed in only one cluster.

The program product of the invention also has means at block 229 for responding to the content of list L and index G to determine whether all genes being analyzed have been placed in a cluster. If rot, the means at block 229 adds each remaining gene to a cluster having a gene with which the remaining gene has a highest match fraction mf regardless of whether mf is less than the threshold mft.

### OPERATION OF THE PREFERRED EMBODIMENT

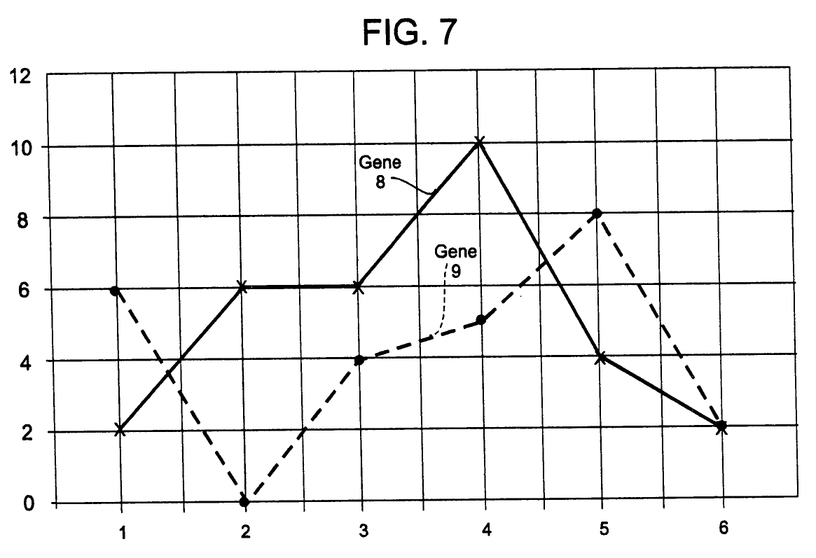

Important features of the invention are that non-statistical clustering is used. This retains the benefits of scale invariance but adds time invariance to the analysis. Unlike other conventional methods, even partial similarity can be recognized. Multiple sub-sequence matches are handled without compromising accuracy and for this reason, the result obtained is very resistant to noise since gaps are allowed. Unlike other methods, the invention allows an algorithm to be used that accommodates a shift in time over which similarity is seen. An example similarity search output is shown below for two hypothetical genes, gene 8 and gene 9 whose profiles are shown in FIG. 7. It can be seen that gene 8 and gene 9 are similar even though they do not overlap exactly because their profiles differ and they are shifted in time.

| SEQ 1 | SEQ 2 | Match Fraction | No. of Subseq |
|---|---|---|---|
| gene 8 | gene 9 | 0.6xxx | 1 |

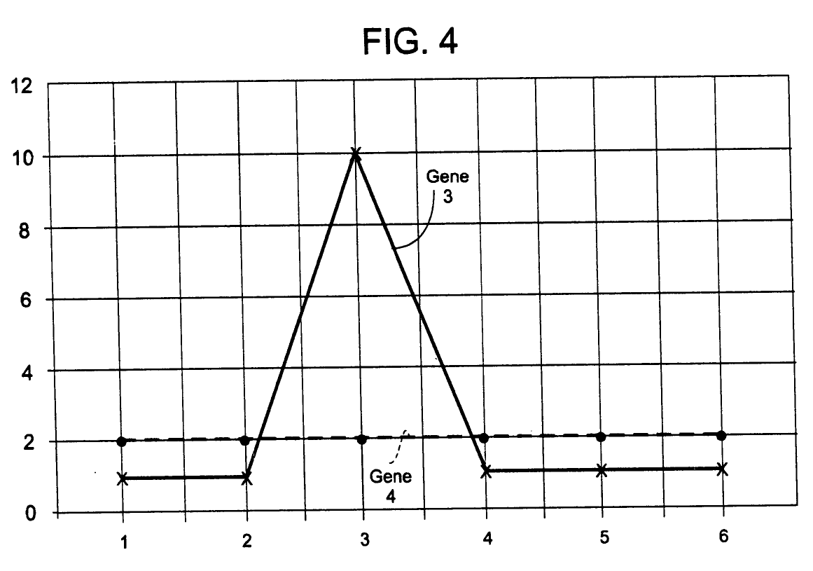

The higher the match fraction, the better is the match of two sequences. The match fraction above is shown for purposes of description only and is not an actual calculated fraction.
Another feature of the algorithm that is accommodated by the method of the invention is that there can be “gaps” in similar sequences as shown in FIG. 4. This may lead to multiple sub-sequence matches and the logic means of the invention handles clustering of such similar sequences without sacrificing accuracy.

The algorithm that is used in this preferred embodiment is described in the paper by R. Agrawal, K. Lin, H. S. Sawhney, K. Shim: “Fast Similarity Search in the Presence of Noise, Scaling, and Translation in Time-Series Databases”, Proc. Of. the 21st Int'l Confererce on Very Large Databases, Zurich, Switzerland, September 1995. This algorithm will be referred to hereinafter as the Agrawal Fast Similarity Search. It is understood that the use of the algorithm per se does not comprise the novelty of the invention but that the novel and unobvious programmed logic means and method of the clustering means permit such use. The algorithm uses a model of similarity of time sequences that presents fast search technique. The amplitude of one of the two sequences is scaled by any suitable amount and its offset is adjusted appropriately. The matching of sequences is then scale-independent, state-independent, translation neutral and noise resistant. The algorithm creates a fast, indexable data structure using small, atomic subsequences that represent all the sequences up to amplitude scaling and offset. R*-tree family of structures are used for this representation because arbitrary precision can be maintained for the sequence values while still allowing for similarities to be defined with respect to a user-defined e distance in L-infinity norm between the atomic subsequences. Therefore, all atomic subsequence matches within a distance e can be efficiently calculated. The second stage employs a fast algorithm for stitching atomic matches to form long subsequence matches, allowing non-matching gaps to exists between the atomic matches. The third stage linearly orders the subsequence matches found in the second stage to determine if enough similar pieces exist in the two sequences.

A typical gene expression data set appears below as table I. g1 to g7 are 7 genes expressed over 6 time stamps. Table I is a data set of gene expression profiles which also appear as data set 211 in FIG. 1.

TABLE I

| Gene | t = 1 | t = 2 | t = 3 | t = 4 | t = 5 | t = 6 |
|---|---|---|---|---|---|---|
| g1 | 0.1 | 0.2 | 0.3 | 0.4 | 0.5 | 0.6 |
| g2 | 1 | 2 | 3 | 4 | 5 | 6 |
| g3 | 1 | 1 | 10 | 1 | 1 | 1 |
| g4 | 2 | 2 | 2 | 2 | 2 | 2 |
| g5 | 1 | 0.8 | 0.6 | 0.4 | 0.2 | 0 |
| g6 | 10 | 10 | 6 | 4 | 2 | 0 |
| g7 | 0 | 0.4 | 0.6 | 0.8 | 1 | 1.2 |

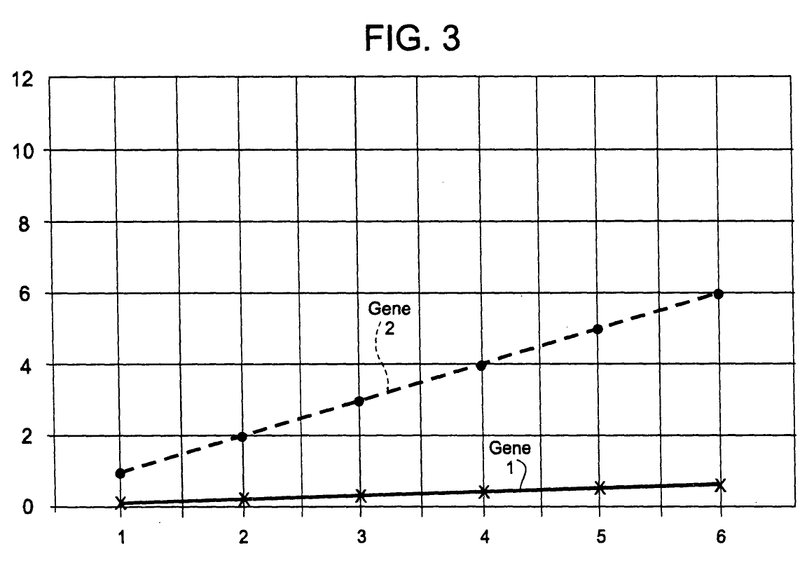

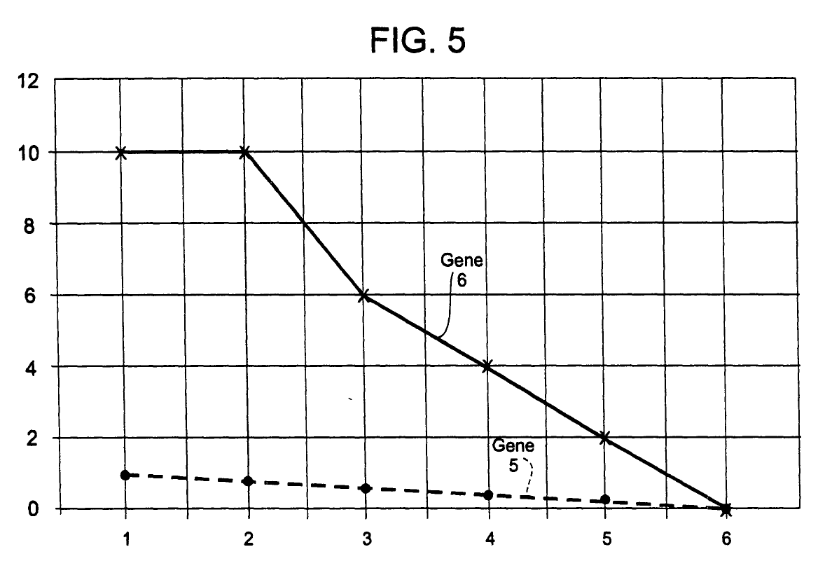

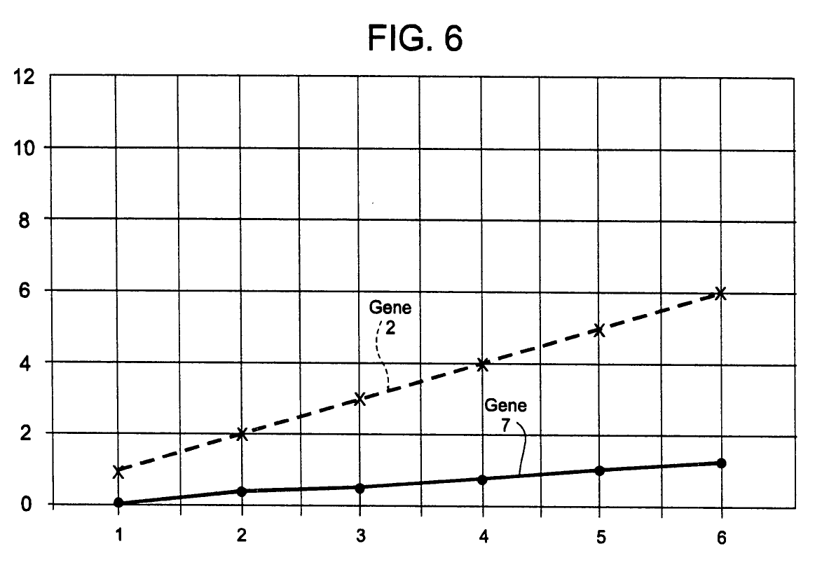

If one plots these 7 lines as a function of time we have the graphs shown in FIGS. 3, 4, 5 and 6.
Referring to FIG. 3, it can be seen that even though gene 1 and gene 2 are in different scale, the underlying trend is the same and hence one would conclude that they are functionally similar.
However, with gene 7 and gene 1, shown in FIG. 6, the match is not so clear since initially, the slope of g7 is steeper. However, if we looked only at a subsequence matching of timestamp t=2 to t−6, the trend is seen to be similar.
This similarity which is shifted in time can be identified by the Agrawal Fast Similarity Search algorithm which identifies these two as similar genes.
FIG. 4 exemplifies noise resistance and partial similarity. When one looks at gene 4 and gene 3, it is clear that most likely, the value of 10 for gene 3 at t=3 is an outlier. This data point could have occurred, either from manual error or instrumentation error. The Agrawal Fast Similarity Search algorithm will minimize this artifact data point by its design, and identify two matching areas. The profile from t=1 to t=2 is identified as one subsequence and the profile from t=4 to t=6 as another subsequence. Since it has minimized this “outlier or noise”, it is able to identify these two genes as similar in function.

These results of similarity are used by the invention for clustering. The data shown in FIG. 1 at 215 is an example data set output of gene expression pairs and a match fraction for each pair from the Intelligent Miner for Data 213 of FIG. 1. The data set of gene expression pairs and a match fraction for each pair shown in table II below is similar to that of block 215 of FIG. 1 but is not listen in descending order of match fraction value to facilitate explanation of another feature of the method of the invention.

TABLE II

| Gene1 | Gene2 | match | subsequences |
|---|---|---|---|
| g1 | g2 | 1 | 1 |
| g1 | g7 | 0.6 | 1 |
| g2 | g7 | 0.6 | 1 |
| g3 | g4 | 0.8 | 2 |
| g5 | g6 | 0.7 | 1 |
| g3 | g6 | 0.2 | 1 |
| g4 | g6 | 0.2 | 1 |
| . | . | . | . |
| . | . | . | . |
| . | . | . | . |

Referring again to FIG. 2, the programmed logic flow begins at block 217 to first create a list L of all of the genes being processed for similarity. This list is used to determine when all genes have been processed. An index array G and an cluster index array C are also set up in block 217. Array G will store the indices of the genes that have been processed at least once in the process of clustering. Array C stores the number of clusters that have been found. Then the program at block 217 accepts a match fraction threshold input mft from a user or from another application program. At this state, the program at block 221 lists those gene pairs of the algorithm output 215 having a match fraction greater than the threshold, into a list 219 in descending order of match fraction value. At block 223 gene expression pairs are listed in clusters by their match fractions.

The logic of block 223 places the genes gi through gene gz into appropriate clusters. For each similar gene pair in 219, if the index i of gi has been seen before as evidenced by an entry i in G, the method skips to the next gene pair. If the index i of gi has not been seen in G but the index j of the other gene of the pair has been seen in G, then the pair gi,gj belong in the cluster ca to which gene gj belongs. If the index i of gi has not been seen in G and index j of the other gene of the pair has also not been seen in G, then the pair gi,gj belong in a new cluster cb. In this way the logic at block 223 lists gene expression pairs in clusters by their match fractions. Another way to express these results is using associative logic. That is if A is similar to B and B is similar to C, then A,B, and C belong to a similar group.

This method is applied to the data of table II. For example gene 1 and gene 2 are the first pair in table II and they are placed in cluster cl as shown below in table III. Gene 1 and gene 7 are the second pair in table II and by the program logic of block 223, gene 7 also is placed in cluster c1.

Thus we have 3 clusters if the threshold is above 0.2 and 4 clusters if no threshold is set or if the threshold is less than 0.2. This result is shown in table form below as table IV.

TABLE IV

| | |
|---|---|
| Cluster 1: | g1, g2, g7 |
| Cluster 2: | g3, g4 |
| Cluster 3: | g5, g6, |
| Cluster 4: | g3, g6, g4. |

Referring now to the table above, gene 3 and gene 4 are seen to each be in two clusters. According to the logic of block 225, the match fraction (0.2) of gene 3 and gene 6 of cluster 4 is compared to the match fraction (0.8) of gene 3 and gene 4 of cluster 3. Since the match fraction (0.8) of cluster 2 is greater than (0.2), gene 3 is removed from cluster 4 and retained in cluster 2. Likewise gene 4 is removed from cluster 4 and retained in cluster 2. Likewise gene 6 is removed from cluster 4 and retained in cluster 3. This logic is expressed in block 225 as: if gene gk belongs to cluster ca and to cluster cb, and the maximum match fraction of gk in cluster ca is greater than the maximum match fraction of gk in cluster cb then the gene gk is placed only in cluster ca.

In an embodiment where a threshold is provided, also shown in FIG. 2, the number of steps of the method can be reduced by providing fewer examples of genes in more than one cluster. In this embodiment we look for similar genes having match fractions higher than a pre-specified minimum threshold. For example, let the minimum threshold be 0.5 then gene 1 and gene 2 are in the same cluster, gene 1 and gene 7 are in one cluster etc. Now we have 3 clusters as shown in table V below:

TABLE V

| | |
|---|---|
| Cluster 1: | g1, g2, g7 |
| Cluster 2: | g3, g4 |
| Cluster 3: | g5, g6. |

Since all other match fractions are less than 0.5, these pairs will not be included in cluster building logic and because all gene expression profiles g1 through g7 have all been accounted for, the process ends.

In another embodiment, we may have identified a particular gene gn of interest contained in a data set 233 shown in FIG. 1 and we are now looking for all genes behaving similarly but they are stored in an different data set 211 that has been created using similar experimental conditions. The steps in this embodiment are shown in FIG. 1 as follows:

First we insert the gene expression for the particular gene of interest as a row gn into the data set 211 of interest. Then we perform the algorithm processing step of block 213 in the method of FIG. 1 described above. The next steps create clusters as shown in FIG. 2. Now the method selects, using scripts or table operations, the cluster that contains gene gn as one of the elements of the cluster.

In a still further embodiment, we have identified a particular set of genes cp=gm, gn, . . . from a data set 233 and we are now looking for all genes behaving similarly but they are stored in an different data set 211 that has been created using similar experimental conditions. The steps in this embodiment are as follows:

First we insert the gene expressions for the particular genes of interest gm, gn, . . . as rows gm, gn, . . . into the data set 211 of interest. Then we perform the algorithm processing step of block 213 in the method of FIG. 1 described above. After creating clusters according to the invention as described with respect to FIG. 2, the method selects, using scripts or table operations, those clusters that contain genes gm, gn, . . . as one of the elements.

Having described the programmed means and method of the invention and several embodiment thereof, it may be seen that the present invention overcomes the shortcomings of the prior art systems by providing clusters of genes in the presence of noise and time shifts by a programmed apparatus using efficient method steps. It will be understood by those skilled in the art of computer systems that many additional modifications and adaptations to the present invention can be made in both embodiment and application without departing from the spirit and scope of this invention. Accordingly, this description should be considered as illustrative of the present invention and not in limitation thereof.

1. A method of determining functional similarity between portions of gene expression profiles comprising the steps of:
processing a number of gene expression profiles with a similar sequences algorithm that is a time and intensity invariant correlation function to obtain a data set of gene expression pairs and a match fraction for each pair;
listing gene expression pairs in clusters by their match fractions;
removing a first gene from a cluster when another cluster has another gene with a higher match fraction with the first gene, unless the another gene requires a larger number of subsequences to achieve similarity with the first gene;
repeating the removing step until all genes are listed in only one cluster.

2. A method of determining functional similarity between portions of gene expression profiles comprising the steps of:
processing a number of gene expression profiles with a similar sequences algorithm that is a time and intensity invariant correlation function to obtain a data set of gene expression pairs and a match fraction for each pair;
listing gene expression pairs in clusters by their match fractions;
removing a first gene from a first cluster when the first gene is also in a second cluster which has another gene with a higher match fraction with the first gene than any of the genes in the first cluster have with the first gene, but;
retaining the first gene in the first cluster and removing the first gene from the second cluster when the difference between the highest match fraction of the first gene with a gene in the first cluster and the highest match fraction of the first gene with a gene in the second cluster is less than a minimum difference threshold and the number of subsequences represented in the similar gene pair having the highest match fraction in the first cluster is higher than the number of subsequences represented in the similar gene pair having the highest match fraction in the second cluster;
repeating the removing step until all genes are listed in only one cluster.

3. A method of determining functional similarity between portions of gene expression profiles comprising the steps of:
processing a number of gene expression profiles with a similar sequences algorithm that is a time and intensity invariant correlation function to obtain a data set of gene expression pairs and a match fraction for each pair;
choosing a threshold match fraction;
listing gene expression pairs in clusters by their match fractions above the threshold;
adding each gene not already in a cluster to a cluster having another gene having a highest match fraction with the each gene without regard of the threshold;
removing a first gene from a cluster when the first gene is also in another cluster which has another gene with a higher match fraction with the first gene than any of the genes in the cluster have with the first gene;
repeating the removing step until all genes are listed in only one cluster.

4. A method of determining functional similarity between portions of gene expression profiles comprising the steps of:
processing a number of gene expression profiles with a similar sequences algorithm that is a time and intensity invariant correlation function to obtain a data set of gene expression pairs and a match fraction for each pair;
choosing a threshold match fraction;
listing gene expression pairs in clusters by their match fractions above the threshold;
adding each gene not already in a cluster to a cluster having another gene having a highest match fraction disregarding the threshold with the each gene;
removing a first gene from a first cluster when the first gene is also in a second cluster which has another gene with a higher match fraction with the first gene than any of the genes in the first cluster have with the first gene, but;
retaining the first gene in the first cluster and removing the first gene from the second cluster when the difference between the highest match fraction of the first gene with a gene in the first cluster and the highest match fraction of the first gene with a gene in the second cluster is less than a minimum difference threshold and the number of subsequences represented in the similar gene pair having the highest match fraction in the first cluster is higher than the number of subsequences represented in the similar gene pair having the highest match fraction in the second cluster;
repeating the removing and retaining steps until all genes are listed in only one cluster.

5. A method of determining functional similarity between genes comprising the steps of:
listing genes to be compared in a data set by their gene expression profiles;
processing the listed gene expression profiles with a similar sequences algorithm that is a time and intensity invariant correlation function to obtain a data set of gene expression pairs and a match fraction for each pair;
choosing a threshold match fraction;
creating a set G in which to list indices of genes accounted for;
assigning genes i and j to a cluster a if they have a match fraction greater than the threshold;
assigning gene k to the cluster a if it has a match fraction greater than the threshold with either gene i or gene assigning genes k and l to a cluster b if they have a match fraction greater than the threshold and if both gene k and gene l do not have match fractions above the threshold with either gene i or gene j;
repeating the assigning steps until all genes to be compared have been considered;
removing a first gene from a cluster when another cluster has another gene with a higher match fraction with the first gene;
repeating the removing step until all genes are listed in only one cluster.

6. A method of determining functional similarity between genes comprising the steps of:
listing genes to be compared in A data set by their gene expression profiles;
processing the listed gene expression profiles with a similar sequences algorithm that is a time and intensity invariant correlation function to obtain a data set of gene expression pairs and a match fraction for each pair;
choosing a threshold match fraction;
creating a set G in which to list indices of genes accounted for;
assigning genes i and j to cluster 1 if they have a match fraction greater than the threshold;
assigning gene k to cluster 1 if it has a match fraction greater than the threshold with either gene i or gene j;
assigning genes k and 1 to cluster 2 if they have a match fraction greater than the threshold and if both gene k and gene 1 do not have match fractions above the threshold with either gene i or gene j;
removing a first gene from a cluster when another cluster has another gene with a higher match fraction with the first gene, unless the another gene requires a larger number of subsequences to achieve similarity with the first gene;
repeating the removing step until all genes are listed in only one cluster.

7. A method of determining functional similarity between a gene of interest gn whose expression profile is contained in a data set and other genes in another data set that has been created using similar experimental conditions comprising the steps of:
inserting a gene expression profile for the gene of interest gn into the another data set;
processing the gene expression profiles of the another data set with a similar sequences algorithm that is a time and intensity invariant correlation function to obtain a data set of gene expression pairs and a match fraction for each pair;
choosing a threshold match fraction;
listing gene expression pairs in clusters by their match fractions above the threshold;
adding each gene not already in a cluster to a cluster having another gene having a highest match fraction with the each gene;
removing a first gene from a cluster when the first gene is also in another cluster which has another gene with a higher match fraction with the first gene than any of the genes in the cluster have with the first gene;
repeating the removing step until all genes are listed in only one cluster.
selecting the cluster that contains gene gn as one of the elements of the cluster.

8. A method of determining functional similarity between a gene of interest gn whose expression profile is contained in a data set and other genes in another data set that has been created using similar experimental conditions comprising the steps of:
inserting a gene expression profile for the gene of interest gn into the another data set;
processing the gene expression profiles of the another data set with a similar sequences algorithm that is a time and intensity invariant correlation function to obtain a data set of gene expression pairs and a match fraction for each pair;
choosing a threshold match fraction;
listing gene expression pairs in clusters by their match fractions above the threshold;
adding each gene not already in a cluster to a cluster having another gene having a highest match fraction with the each gene;
removing a first gene from a cluster when the first gene is also in another cluster which has another gene with a higher match fraction with the first gene than any of the genes in the cluster have with the first gene, unless the another gene requires a larger number of subsequences to achieve similarity with the first gene;
repeating the removing step until all genes are listed in only one cluster.
selecting the cluster that contains gene gn as one of the elements of the cluster.

9. A method of determining functional similarity between a particular set of genes of interest cp whose expression profiles are contained in a data set and other genes in another data set that has been created using similar experimental conditions comprising the steps of:
inserting a gene expression profile for each gene of interest in the set of genes of interest into the another data set;
processing the gene expression profiles of the another data set with a similar sequences algorithm that is a time and intensity invariant correlation function to obtain a data set of gene expression pairs and a match fraction for each pair;
choosing a threshold match fraction;
listing gene expression pairs in clusters by their match fractions above the threshold;
adding each gene not already in a cluster to a cluster having another gene having a highest match fraction with the each gene;
removing a first gene from a cluster when the first gene is also in another cluster which has another gene with a higher match fraction with the first gene than any of the genes in the cluster have with the first gene;
repeating the removing step until all genes are listed in only one cluster.
selecting those clusters that contains a gene from the set of genes of interest as one of the elements of the cluster.

10. A program product having computer readable code stored on a recordable media for determining functional similarity between portions of gene expression profiles comprising:
programmed means for processing a number of gene expression profiles with a similar sequences algorithm that is a time and intensity invariant correlation function to obtain a data set of gene expression pairs and a match fraction for each pair;
programmed means for listing gene expression pairs in clusters by their match fractions;
programmed means for removing a first gene from a cluster when the first gene is also in another cluster which has another gene with a higher match fraction with the first gene than any of the genes in the cluster have with the first gene;
programmed means for repeating the removing step until all genes are listed in only one cluster.

11. A program product having computer readable code stored on a recordable media for determining functional similarity between portions of gene expression profiles using output from a similar sequences algorithm that is a time and intensity invariant correlation function comprising:
programmed means for providing a gene expression profile data set as input to programmed means embodying a similar sequences algorithm that is a time and intensity invariant correlation function to obtain a data set of gene expression pairs and a match fraction for each pair as output from the programmed means embodying a similar sequences algorithm;
programmed means for listing the gene expression pairs in clusters by their match fractions;
programmed means for removing a first gene from a cluster when the first gene is also in another cluster which has another gene with a higher match fraction with the first gene than any of the genes in the cluster have with the first gene;
programmed means for repeating the removing step until all genes are listed in only one cluster.

12. A program product having computer readable code stored on a recordable media for determining functional similarity between portions of gene expression profiles comprising the steps of:
programmed means for processing a number of gene expression profiles with a similar sequences algorithm that is a time and intensity invariant correlation function to obtain a data set of gene expression pairs and a match fraction for each pair;
programmed means for listing gene expression pairs in clusters by their match fractions;
programmed means for removing a first gene from a first cluster when the first gene is also in a second cluster which has another gene with a higher match fraction with the first gene than any of the genes in the first cluster have with the first gene, but;
programmed means for retaining the first gene in the first cluster and removing the first gene from the second cluster when the difference between the highest match fraction of the first gene with a gene in the first cluster and the highest match fraction of the first gene with a gene in the second cluster is less than a minimum difference threshold and the number of subsequences represented in the similar gene pair having the highest match fraction in the first cluster is higher than the number of subsequences represented in the similar gene pair having the highest match fraction in the second cluster;
programmed means for repeating the removing step until all genes are listed in only one cluster.

13. A program product having computer readable code stored on a recordable media for determining functional similarity between portions of gene expression profiles comprising the steps of:
programmed means for processing a number of gene expression profiles with a similar sequences algorithm that is a time and intensity invariant correlation function to obtain a data set of gene expression pairs and a match fraction for each pair;
programmed means for choosing a threshold match fraction;
programmed means for listing gene expression pairs in clusters by their match fractions above the threshold;
programmed means for adding each gene not already in a cluster to a cluster having another gene having a highest match fraction with the each gene without regard of the threshold;
programmed means for removing a first gene from a cluster when the first gene is also in another cluster which has another gene with a higher match fraction with the first gene than any of the genes in the cluster have with the first gene;
programmed means for repeating the removing step until all genes are listed in only one cluster.

14. A program product having computer readable code stored on a recordable media for determining functional similarity between portions of gene expression profiles comprising the steps of:
programmed means for processing a number of gene expression profiles with a similar sequences algorithm that is a time and intensity invariant correlation function to obtain a data set of gene expression pairs and a match fraction for each pair;
programmed means for choosing a threshold match fraction;
programmed means for listing gene expression pairs in clusters by their match fractions above the threshold;
programmed means for adding each gene not already in a cluster to a cluster having another gene having a highest match fraction disregarding the threshold with the each gene;
programmed means for removing a first gene from a first cluster when the first gene is also in a second cluster which has another gene with a higher match fraction with the first gene than any of the genes in the first cluster have with the first gene, but;
programmed means for retaining the first gene in the first cluster and removing the first gene from the second cluster when the difference between the highest match fraction of the first gene with a gene in the first cluster and the highest match fraction of the first gene with a gene in the second cluster is less than a minimum difference threshold and the number of subsequences represented in the similar gene pair having the highest match fraction in the first cluster is higher than the number of subsequences represented in the similar gene pair having the highest match fraction in the second cluster;
programmed means for repeating the removing and retaining steps until all genes are listed in only one cluster.

15. A program product having computer readable code stored on a recordable media for determining functional similarity between genes comprising the steps of:
programmed means for listing genes to be compared by their gene expression profiles;
programmed means for processing the listed gene expression profiles with a similar sequences algorithm that is a time and intensity invariant correlation function to obtain a data set of gene expression pairs and a match fraction for each pair;
programmed means for choosing a threshold match fraction;
programmed means for creating a null set G(0) to hold genes accounted for;
programmed means for assigning genes i and j to cluster 1 if they have a match fraction greater than the threshold;
programmed means for assigning gene k to cluster 1 if it has a match fraction greater than the threshold with either gene i or gene j;
programmed means for assigning genes k and 1 to cluster 2 if they have a match fraction greater than the threshold and if both gene k and gene 1 do not have match fractions above the threshold with either gene i or gene j;
programmed means for removing a first gene from a cluster when another cluster has another gene with a higher match fraction with the first gene;
programmed means for repeating the removing step until all genes are listed in only one cluster.

16. A program product having computer readable code stored on a recordable media for determining functional similarity between genes comprising the steps of:
programmed means for listing genes to be compared by their gene expression profiles;
programmed means for processing the listed gene expression profiles with a similar sequences algorithm that is a time and intensity invariant correlation function to obtain a data set of gene expression pairs and a match fraction for each pair;
programmed means for choosing a threshold match fraction;
programmed means for creating a null set G(0) to hold genes accounted for;
programmed means for assigning genes i and j to cluster 1 if they have a match fraction greater than the threshold;
programmed means for assigning gene k to cluster 1 if it has a match fraction greater than the threshold with either gene i or gene j;
programmed means for assigning genes k and 1 to cluster 2 if they have a match fraction greater than the threshold and if both gene k and gene 1 do not have match fractions above the threshold with either gene i or gene j;
programmed means for removing a first gene from a cluster when another cluster has another gene with a higher match fraction with the first gene, unless the another gene requires a larger number of subsequences to achieve similarity with the first gene;
programmed means for repeating the removing step until all genes are listed in only one cluster.

17. A program product having computer readable code stored on a recordable media for determining functional similarity between a gene of interest gn whose expression profile is contained in a data set and other genes in another data set that has been created using similar experimental conditions comprising the steps of:
programmed means for inserting a gene expression profile for the gene of interest gn into the another data set;
programmed means for processing the gene expression profiles of the another data set with a similar sequences algorithm that is a time and intensity invariant correlation function to obtain a data set of gene expression pairs and a match fraction for each pair;
programmed means for choosing a threshold match fraction;
programmed means for listing gene expression pairs in clusters by their match fractions above the threshold;
programmed means for adding each gene not already in a cluster to a cluster having another gene having a highest match fraction with the each gene;
programmed means for removing a first gene from a cluster when the first gene is also in another cluster which has another gene with a higher match fraction with the first gene than any of the genes in the cluster have with the first gene;
programmed means for repeating the removing step until all genes are listed in only one cluster.
programmed means for selecting the cluster that contains gene gn as one of the elements of the cluster.

18. A program product having computer readable code stored on a recordable media for determining functional similarity between a gene of interest gn whose expression profile is contained in a data set and other genes in another data set that has been created using similar experimental conditions comprising the steps of:
programmed means for inserting a gene expression profile for the gene of interest gn into the another data set;
programmed means for processing the gene expression profiles of the another data set with a similar sequences algorithm that is a time and intensity invariant correlation function to obtain a data set of gene expression pairs and a match fraction for each pair;
programmed means for choosing a threshold match fraction;
programmed means for listing gene expression pairs in clusters by their match fractions above the threshold;
programmed means for adding each gene not already in a cluster to a cluster having another gene having a highest match fraction with the each gene;
programmed means for removing a first gene from a cluster when the first gene is also in another cluster which has another gene with a higher match fraction with the first gene than any of the genes in the cluster have with the first gene, unless the another gene requires a larger number of subsequences to achieve similarity with the first gene;
programmed means for repeating the removing step until all genes are listed in only one cluster.
programmed means for selecting the cluster that contains gene gn as one of the elements of the cluster.

19. A program product having computer readable code stored on a recordable media for determining functional similarity between a particular set of genes of interest cp whose expression profiles are contained in a data set and other genes in another data set that has been created using similar experimental conditions comprising the steps of:
programmed means for inserting a gene expression profile for each gene of interest in the set of genes of interest into the another data set;
programmed means for processing the gene expression profiles of the another data set with a similar sequences algorithm that is a time and intensity invariant correlation function to obtain a data set of gene expression pairs and a match fraction for each pair;
programmed means for choosing a threshold match fraction;
programmed means for listing gene expression pairs in clusters by their match fractions above the threshold;
programmed means for adding each gene not already in a cluster to a cluster having another gene having a highest match fraction with the each gene;
programmed means for removing a first gene from a cluster when the first gene is also in another cluster which has another gene with a higher match fraction with the first gene than any of the genes in the cluster have with the first gene;
programmed means for repeating the removing step until all genes are listed in only one cluster.
programmed means for selecting those clusters that contains a gene from the set of genes of interest as one of the elements of the cluster.

20. In a method of determining functional similarity between portions of gene expression profiles which includes processing a number of gene expression profiles with a similar sequences algorithm that is a time and intensity invariant correlation function to obtain a data set of gene expression pairs and a match fraction for each pair, the improvement comprising the steps of:
listing gene expression pairs in clusters by their match fractions;
removing a first gene from a cluster when another cluster has another gene with a higher match fraction with the first gene, unless the another gene requires a larger number of subsequences to achieve similarity with the first gene;
repeating the removing step until all genes are listed in only one cluster.
